(19) United States
(12) Patent Application Publication
(10) Pub. No.: US 2008/0254023 A1
(43) Pub. Date: Oct. 16, 2008

(54) Treating Gliosis, Glial Scarring, Inflammation or Inhibition of Axonal Growth in the Nervous System by Modulating Eph Receptor

(75) Inventors: Perry F. Bartlett, Queensland (AU); Mary P. Galea, Queensland (AU); Yona Goldsmith, Victoria (AU); Ann M. Turnley, Victoria (AU); Andrew W. Boyd, Queensland (AU)

Correspondence Address: FOLEY AND LARDNER LLP;SUITE 500, 3000 K STREET NW, WASHINGTON, DC, 20007, US

(73) Assignees: THE UNIVERSITY OF QUEENSLAND, Queensland (AU); THE UNIVERSITY OF MELBOURNE, Victoria (AU)

(21) Appl. No.: 11/662,355

(22) PCT Filed: Sep. 8, 2005
(86) PCT No.: PCT/AU05/01363

§ 371 Date: May 8, 2008

Related U.S. Application Data
(60) Provisional application No. 60/647,968, filed on Jan. 27, 2005.

(30) Foreign Application Priority Data
Sep. 8, 2004 (AU) 2004905148

(51) Int. Cl. A61K 39/395; A61K 38/02; A61P 25/00; G01N 33/53; A61K 31/70

(52) U.S. Cl. 424/133.1; 514/2; 514/44; 424/130.1; 435/7.21

(57) ABSTRACT

The present invention relates to a method of treating disorders of the nervous system and more particularly disorders associated with a gliotic response and/or an inflammatory response within the central nervous system and to therapeutic agents useful for same. More particularly, the present invention involves a method of preventing or reducing the amount of Eph receptor-mediated gliosis and/or glial scarring and/or inflammation and/or Eph receptor-mediated inhibition of axonal growth which occurs during and/or after disease or injury to the nervous system. The present invention also facilitates the identification of therapeutic agents which modulate Eph receptor-mediated signaling. The method and therapeutic agents of the present invention are useful for treating a range of nervous system diseases, conditions and injuries including, inter alia, paralysis induced by physiological-, pathological- or trauma-induced injury to the brain or spinal cord.

### BACKGROUND OF THE INVENTION

1. Field of the Invention

The present invention relates to a method of treating disorders of the nervous system and more particularly disorders associated with a gliotic response and/or an inflammatory response within the central nervous system and to therapeutic agents useful for same. More particularly, the present invention involves a method of preventing or reducing the amount of Eph receptor-mediated gliosis and/or glial scarring and/or inflammation and/or Eph receptor-mediated inhibition of axonal growth which occurs during and/or after disease or injury to the nervous system. The present invention also facilitates the identification of therapeutic agents which modulate Eph receptor-mediated signaling. The method and therapeutic agents of the present invention are useful for treating a range of nervous system diseases, conditions and injuries including, inter alia, paralysis induced by physiological-, pathological- or trauma-induced injury to the brain or spinal cord.

2. Description of the Prior Art

Reference to any prior art in this specification is not, and should not be taken as, an acknowledgment or any form of suggestion that this prior art forms part of the common general knowledge in any country.

Bibliographic details of references provided in this document are listed at the end of the specification.

The nervous system, especially the central nervous system, exhibits a limited capacity to regenerate after disease or injury. In many cases, the damage caused by a disease or injury to the central nervous system results in permeant mental and/or physical disablement. In addition to the significant personal suffering that disablement causes to people, diseases and injuries of the central nervous system cost society billions of dollars per year in treatment, rehabilitation and sustained welfare.

One of the major factors underlying the lack of repair in the central nervous system is the inability of axons to regenerate through areas of damage. One possible explanation for this is the presence of proteins in the area of damage that inhibit the re-growth of axons. Indeed, proteins such as Nogo (Bandtlow and Schwab, Glia 29:175-181, 2000; Chen et al., Nature 403:434-439, 2000), myelin-associated-glycoprotein (MAG; McKerracher et al., Neuron 13:805-811, 1994; Mukhopadhyay et al., Neuron 13:757-767, 1994) and certain chondroitin sulfate proteoglycans (Dou et al., J Neurosci 14:7616-7628, 1994; Stichel et al., Eur J Neurosci 7:401-411, 1995; Niederost et al., J Neurosci 19:8979-8989, 1999) have all been shown to posses significant axon growth inhibitory properties. However, recent studies have shown that blocking or deleting these proteins leads to minor or even non-detectable improvements in axonal regeneration (Kim et al., Neuron 38:187-199, 2003; Simonen et al., Neuron 38:201-211, 2003; Zheng et al., Neuron 38:187-199, 2003).

Another possible explanation for the lack of axonal regeneration through areas of the central nervous system damaged by disease or injury is the glial scar. The glial scar is a dense mechanical and probably biochemical barrier for regenerating axons that forms at sites of neural damage (Stichel and Muller, Cell Tissue Res 294:1-9, 1998). The scar consists of reactive astrocytes, microglia, oligodendorcytes precursors, and often, fibroblasts. Furthermore, the glial scar also serves as source of inhibitory factors such as those described above (McKeon et al., J Neurosci 11:3398-3411, 1991; Stichel et al., Eur J Neurosci 11:632-646, 1999). Some studies have suggested that glial scar formation may be regulated by inflammatory cytokines (Balasingam et al., J Neurosci 14:846-856, 1994).

A family of molecules known to inhibit the growth of axons are the erythropoietin-producing-hepatoma cell line (Eph) family of receptor tyrosine kinases and their associated ligands, the Eph family receptor interacting proteins (ephrins). The Ephs and ephrins comprise a major group of axonal guidance molecules which are required, inter alia, for the correct development of axonal connections in a number of neural systems (Flanagan and Vanderhaeghen, Ann Rev Neurosci 21:309-345, 1998; Holder and Klein, Development 126:2033-2044, 1999; O'Leary and Wilkinson, Curr Opin Neurobiol 9:65-73, 1999; Nakamoto, Int J Biochem Cell Biol 32:7-12, 2000). Members of the Eph/ephrin families frequently exhibit a dynamic and spatially restricted expression pattern within the developing central nervous system (Mori et al., Brain Res Mol Brain Res 29:325-335, 1995; Kilpatrick et al., Mol Cell Neurosci 7:62-74, 1996; Martone et al., Brain Res 771:238-250, 1997; Connor et al, Dev Biol 193:21-35, 1998; Iwamasa et al., Dev Growth Diff 41:685-698, 1999; Imondi et al., Development 127:1397-1410, 2000; Kury et al., Mol Cell Neurosci 15:123-140, 2000). Currently there are at least 14 known Eph receptors and 8 ephrin ligands (Eph Nomenclature Committee, Cell 90:403-404, 1997). All of the ligands are membrane-bound and are divided into two groups, ephrin-A and ephrin-B, based on structure and function. The ephrin-A ligands are attached to the cell membrane via a glycosylphoshpatidylinositol (GPI) anchor, whereas ephrin-B ligands have a transmembrane domain and cytoplasmic region.

Eph receptors have also been divided into two groups defined as EphA and EphB, according to sequence homology. Generally, EphA receptors bind ephrin-A ligands and EphB receptors bind ephrin-B ligands but EphA4 is an exception as it binds not only ephrin-A ligands but also ephrinB2 and ephrinB3 (Gale et al., Oncogene 13:1343-1352, 1996; Bergemann et al., Oncogene 16:471-480, 1998). It has been hypothesized that ephrins define inhibitory territories of axonal innervation via a contact-dependent repulsive mechanism that is initiated by ephrins binding to Eph receptors (Flanagan and Vanderhaeghen, supra; Kalo and Pasquale, Cell Tissue Res 298:1-9, 1999; Mueller, Ann Rev Neurosci 22:351-388, 1999). Recently, however, members of both the ephrin groups have also been demonstrated to act as receptors by transducing signals upon activation by their cognate Eph receptors (Holland et al., Nature 383:722-725, 1996; Bruckner et al., Science 275:1640-1643, 1997; Davy et al., Genes Dev 13:3125-3135, 1999).

Given the debilitating nature of nervous system diseases, conditions and injuries, there is a need to identify agents which have the potential to act as therapeutic or prophylactic agents.

### SUMMARY OF THE INVENTION

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Abbreviations used herein are defined in Table 1.

The present invention is predicated in part on the determination that gliosis and/or glial scarring and/or inflammation in the nervous system and in particular central nervous system after disease or injury is mediated by an Eph receptor and that decreasing the levels of Eph-mediated signaling at the site of a neural injury or disease can prevent or decrease gliosis and/or glial scarring and/or inflammation. Preventing or decreasing gliosis and/or glial scarring and/or inflammation facilitates axonal regeneration in the central nervous system. In addition, or alternatively, antagonizing the Eph receptor is proposed to physically prevent inhibition of axonal growth. The determination that gliosis and/or glial scarring and/or inflammation is regulated by an Eph receptor and in particular EphA4 facilitates, therefore, the development of a method of treating disorders of the nervous system such as those which arise during, or from, various diseases, conditions or injuries including, inter alia, paralysis induced by physiological-, pathological- or trauma-induced injury to the brain or spinal cord or stroke and the development of therapeutic agents useful for same. Accordingly, the Eph receptor and its ligands are proposed to be suitable targets for agents which prevent or reduce Eph receptor-mediated gliosis and/or glial scarring and/or inflammation.

In one embodiment, therefore, the present invention contemplates a method of preventing or reducing the amount of gliosis and/or glial scarring and/or inflammation in the nervous system said method comprising decreasing the level and/or function of an Eph receptor, or a molecule required for Eph receptor function, in order to decrease levels of Eph receptor-mediated signaling.

Generally, decreasing the level and/or function of the Eph receptor is through the administration to a subject of an agent which prevents Eph receptor-mediated signaling and/or interaction with neuritis.

In yet another embodiment, the present invention provides agents in the form of antagonists of Eph-mediated signaling which are useful for decreasing levels of Eph receptor-mediated signaling at the site of a neural injury or disease. The agents may be any proteinaceous molecules or such as peptides, polypeptides, proteins, antibodies or non-proteinaceous molecules such as nucleic acid molecules and small to medium chemical molecules.

Preferably, the Eph receptor is the EphA4 receptor.

The present invention also provides for methods of identifying agents. These methods for identification comprise screening naturally produced libraries, chemical molecule libraries as well as combinatorial libraries, phage display libraries and in vitro translation-based libraries.

In still yet another embodiment, the present invention provides a method of preventing or reducing the amount of gliosis and/or glial scarring and/or inflammation and/or inhibition of axonal growth in the nervous system of a subject said method comprising administering to said subject an effective amount of an antagonist of EphA4-mediated signaling for a time and under conditions sufficient to prevent or decrease gliosis and/or glial scar formation and/or inflammation.

The antagonists of EphA4-mediated signaling may be administered alone or co-administered in combination with other agents such as agents which promote neurogenesis and/or axon growth and/or inhibit inflammation. Broad or narrow specific agents which antagonize one or more inflammatory cytokines are particularly contemplated by the present invention to be used alone or in combination with EphA4 antagonists.

The present invention also provides pharmaceutical compositions useful for preventing or reducing the amount of gliosis and/or glial scarring and/or inflammation in the nervous system of a subject.

In still yet another embodiment, the present invention provides a method of treating a range of nervous system diseases, conditions and injuries in a subject said method comprising administering to said subject an effective amount of an antagonist of EphA4-mediated signaling for a time and under conditions sufficient to treat said nervous system diseases, conditions and injuries.

TABLE 1
ABBREVIATIONS

| ABBREVIATION | DESCRIPTION |
|---|---|
| Eph | Erythropoietin-producing-hepatoma cell line |
| ephrin | Eph family receptor interacting proteins |
| MAG | Myelin-associated-glycoprotein |
| LIF | Leukemia inhibitory factor |
| IFNγ | Interferon-γ |
| GFAP | Glial fibrillary acidic protein |
| TMRD | Tetramethylrhodamine dextran |

### BRIEF DESCRIPTION OF THE FIGURES

FIG. 1 is a photographical representation showing at 6 days post spinal cord injury (SCI), EphA4−/− axons approach but do not cross the lesion site. Anterograde tracing and confocal analysis of lesioned EphA4−/− spinal cords 6 days after hemisection (a) show large numbers of labeled axons 2.5 mm proximal to the lesion (panel ia) and a small number of axons with growth cones (panel a iii; arrows) approaching the lesion site, which is indicated by the dotted line (i) and shown more clearly in a hematoxylin and eosin (H&E) stained section (ii). (b) Wildtype spinal cord also shows very few axons approaching the lesion site. Panel (b ia) shows labeling 2.5 mm upstream of the lesion site. Panel (b iii) an enlargement of panel (b i) shows few axons upstream of the lesion site. In both panels rostral is to the right and caudal to the left, and the lesion site is indicated by dotted lines. Enlarged areas are indicated by boxed areas and arrows. Scale bar in i 250 μm, ii 200 μm, iii 50 μm.

FIG. 2 is a photographic representation showing extensive axonal regeneration in EphA4−/− mice at 6 weeks post injury. Anterograde tracing and confocal analysis of lesioned EphA4−/− spinal cords 6 weeks after hemisection showed that a large percentage of EphA4−/− axons crossed the lesion site (a, c) and extended caudally (*p<0.001), unlike wildtype (EphA4+/+) axons which did not cross the lesion site (b, c). A montage of confocal images of EphA4−/− spinal cord (a i) showed that the regenerating axons passed through the lesion site (indicated by dotted line and by H&E stained section in (a ii) and extended caudally in a straight line with some “waviness” seen immediately post-lesion (panels a iii, iv and v). In both panels rostral is to the right and caudal to the left, and the lesion site is indicated by dotted lines. Enlarged areas are indicated by boxed areas and arrows. Panel (ii) in both cases shows an adjacent H&E stained section demonstrating the lesion site. Scale bars for panels (i) 250 μm; panels (ii) 200 μm, panels (iii to v) 50 μm. Asterisk in panel (a i) indicates the midline.

FIG. 3 is a photographic and graphical representation showing EphA4 (−/−) mice show multiple tract regeneration and improved function. Identification of regenerating neuronal populations was determined by retrograde tracing using Fast Blue (a-c) and each neuron was plotted using an MD3 microscope digitizer and MD-plot software. Unlike lesioned wildtype (WT) mice (b), multiple axonal tracts regenerated in the lesioned EphA4−/− (KO) mice (a, b), with a pattern similar to that of unlesioned controls (c). Regenerated neurons included corticospinal neurons in layer 5 of the cortex (ai, b), rubrospinal neurons in the red nucleus (RN) (aii, b), as well as neurons in the hypothalamus (Hyp), the vestibular (VN) and reticular nuclei and the periaqueductal grey (PAG) matter. Scale bars in a, 200 μm. Functional analysis of lesioned mice showed that EphA4−/− mice recovered substantial function within 1 month. One day (1 d) after lesion stride length (d), hindpaw grasping (e) and the ability to walk on a horizontal or angled (75°) grid (f) were minimal. Stride length was regained in KO mice within 3 weeks, while wildtype mice reached a plateau at 70% recovery. Grasping and grid-walking were significantly (*p<0.001, n=5 WT and 7 KO mice) improved in KO compared with WT by 1 month, continuing to improve up to 3 months.

FIG. 4 is a photographic representation showing astrocytic gliosis and the glial scar are greatly diminished in EphA4−/− mice following injury. Immunostaining for GFAP expression at the lesion site 4 days following spinal cord lesion showed a florid astrocytic gliosis in wildtype mice (a) which was virtually absent in EphA4−/− mice (d). Under higher magnification, the vast majority of astrocytes in wildtype mice were revealed to be hypertrophic (white arrows) (b, g), unlike EphA4−/− astrocytes (black arrows) (e, g) (*p<0.0001). The total number of astrocytes increased with time post-lesion, with greater numbers in EphA4+/+ spinal cords (h). Immunostaining for chondroitin sulphate proteoglycan, a component of the glial scar, 6 weeks post-lesion, revealed that the scar was diminished in the EphA4−/− mice (f) compared with the wildtype animals (c). Scale bars in panels (a, d) represent 200 μm; in (b, e) 50 μm; in (c, f) 200 μm.

FIG. 5 is a photographic representation showing expression of EphA4 on astrocytes inhibits neurite outgrowth. Following spinal hemisection EphA4 (a) and GFAP (b) are co-expressed as assessed by immunofluorescence on reactive astrocytes at the lesion site (c; a merged image of a and b). EphA4 was also expressed on some neurons (arrow in a-c). Western blot analysis (d) showed upregulation and phosphorylation of EphA4 (p-EphA4) at the lesion site (les) in comparison with unlesioned control (con) mice; * shows a non-specific band present in all lanes. β-actin was used as a loading control and EphA4−/− spinal cord as an EphA4 expression control. The EphA4 expression on astrocytes was inhibitory to cortical neuronal neurite outgrowth, as βIII-tubulin positive cortical neurons on EphA4-expressing (EphA4+/+) astrocytes (e, g) had significantly (*p<0.0001) shorter neurites than on EphA4−/− astrocytes (f, g) after 22 hrs. EphA4−/− neurite outgrowth was also enhanced on EphA4−/− and EphA4+/+ astrocytes, compared with that of wildtype neurons (g; **p<0.0001). The inhibition of neurite outgrowth by EphA4 on astrocytes could be blocked in a dose-dependent manner by addition of monomeric EphrinA5-Fc, but this had no effect on neurites grown on laminin (h). Multimerized (multi) EphrinA5-Fc inhibited neurite outgrowth both on astrocytes and on laminin. Scale bars in (a-c and e, f), 50 μm.

FIG. 6 is a photographic and graphical representation showing (a) Expression of EphA4 was upregulated in cultured astrocytes after 72 hrs by IFNγ and LIF but not TNFα or Il-1, compared with untreated controls (con). These cytokines also induced EphA4 phosphorylation (p-EphA4), similar to EphrinA5-Fc (A5). (b) EphA4 phosphorylation leads to activation of Rho (RhoGTP), a cytoskeletal regulator. Rho was activated at the lesion site in wildtype but not EphA4−/− lesioned spinal cords (L1, L2), while (c) in culture, IFNγ, which is an inducer of astrocytic gliosis, activated Rho in wildtype but not EphA4−/− astrocytes. (d) An in vitro astrocyte proliferation assay showed that under basal conditions (con) both wildtype (WT) and EphA4−/− (KO) astrocytes proliferated similarly over 72 hrs. WT astrocytes showed increased proliferation in response to LIF (*P<0.001) and IFNγ (*P<0.005; **P<0.05), while the EphA4−/− astrocyte response to these factors was markedly decreased and only significant for LIF at 72 hr (*P<0.005). Results are representative of n=3 separate experiments.

FIG. 7 is a photographic and graphical representation showing astrogliosis at the lesion site 4 days after SCI. (A) Compared to PBS injection (A and C), astrogliosis in animals subjected to ephrinA5-Fc injection is significantly reduced (B and D). (B) Compared to PBS injection, the total number of astrocytes/mm² is significantly reduced in animals subjected to ephrinA5-Fc injection at the site of SCI.

FIG. 8 is a photographical representation showing that compared to PBS injections, ephrinA5-Fc injections for 2 weeks inhibits EphA4 upregulation at the lesion site 14 days after SCI.

FIG. 9 is a photographical representation showing ephrinA5-Fc injections for 2 weeks increases axonal regeneration at the lesion site 14 days after SCI.

FIG. 10 is a photographical representation showing PBS injections for 2 weeks does not increase axonal regeneration at the lesion site 14 days after SCI when compared to animals injected with ephrinA5-Fc (FIG. 9).

FIG. 11 is a graphical representation showing improvement in grid walking and climbing 4 weeks after SCI in animals injected with ephrinA5-Fc.

FIG. 12 is a photographical representation showing ephrinA5-Fc injections for 2 weeks significantly increases axonal regeneration at the lesion site 6 weeks after SCI.

### DETAILED DESCRIPTION OF THE INVENTION

The present invention is predicated in part on the identification that glial scar formation in the central nervous system after disease or injury is mediated by an Eph receptor and in particular Eph-mediated signaling. The determination that glial scar formation is regulated by an Eph receptor facilitates the development of a method of treating disorders of the nervous system such as those which arise during, or from, disease or injury and therapeutic agents useful for same.

Prior to describing the present invention in detail, it is to be understood that unless otherwise indicated, the subject invention is not limited to specific therapeutic components, manufacturing methods, dosage regimens, or the like, as such may vary. It is also to be understood that the terminology used herein is for the purpose of describing particular embodiments only and is not intended to be limiting.

It must also be noted that, as used in the subject specification, the singular forms “a”, “an” and “the” include plural aspects unless the context clearly dictates otherwise. Thus, for example, reference to “an Eph receptor” includes a single Eph receptor, as well as two or more Eph receptors; reference to “a therapeutic agent” includes a single therapeutic agent, as well as two or more therapeutic agents; and so forth.

The term “gliosis” includes any condition resulting in a gliotic response including inhibition of axon growth.

Reference herein to “glosis” means a substantial amount of glial cell proliferation and/or glial hypertrophy and/or expression of specific markers such as GFAP and/or CSPG. Reference herein to “glial cell” means a reference to any cell of glial lineage such as, but not limited to, astrocytes, oligodendrocytes, Schwann cells and microglia. This may result in one embodiment formation of a glial scar which is an area of the nervous system and inhibits the subsequent regeneration of axons by either physically inhibiting the growth of axons, or, by releasing inhibitory factors which inhibit the growth of axons through a variety of biological mechanisms.

In one embodiment, the present invention provides a method of preventing or reducing the amount of gliosis and/or glial scarring and/or inflammation inhibition of axonal growth in the nervous system of a subject said method comprising administering to said subject an agent which decreases the level and/or function of an Eph receptor, or a molecule required for Eph receptor function, in order to decrease levels of Eph receptor-mediated signaling.

Reference herein to “Eph receptor” means any receptor which is a member of the Eph family of receptor tyrosine kinases such as, but not limited to, EphA1, EphA2, EphA3, EphA4, EphA5, EphA6, EphA7, EphA8, EphB1, EphB2, EphB3, EphB4, EphB5 and EphB6. Preferably, the Eph receptor of the present invention is a member of the EphA group of Eph receptors. The most preferred Eph receptor of the present invention is EphA4.

Accordingly, in another embodiment, the present invention provides a method of preventing or reducing the amount of gliosis and/or glial scarring and/or inflammation and/or inhibition of axonal growth in the nervous system of a subject said method comprising administering to said subject an agent which decrease the expression and/or function of an EphA4 receptor, or a molecule required for normal EphA4 receptor function, in order to decrease levels of EphA4 receptor-mediated signaling.

Reference herein to “EphA4” includes reference to all forms of EphA4 such as EphA4 homologs, paralogs, orthologs, derivatives, fragments and functional equivalents.

Reference to a “subject” includes a human as well as a non-human primate, a laboratory test animal, companion animal or wild animal. Preferably, the subject is a human.

The present invention may also be practiced by modulating levels of a ligand for the EphA4 receptor i.e. an ephrin, or a molecule required for normal ephrin function. Particularly preferred ephrins are those ephrins which functionally interact with EphA4 such as, but not limited to, ephrinA2, ephrinA3, ephrinA4, ephrinA5, ephrinA6, ephrinB1, ephrinB2 and ephrinB3. Reference herein to “functionally interact” means to bind to an Eph receptor where binding results in the activation of the Eph receptor and the elicitation of a biological response. Reference herein to “ephrin” includes reference to all forms of an ephrin such as ephrin homologs, paralogs, orthologs, derivatives, fragments and functional equivalents. In addition or alternatively, the Eph receptor antagonist may prevent interaction with neuritis therefore leading to inhibition of axon growth.

Levels of EphA4 and ephrin ligand may be modulated in accordance with the present invention by an agent. In addition, kinase activity or levels or other components in a downstream signaling pathway may also be modulated by the agent. The “agent” may also be referred to as a therapeutic agent, therapeutic molecule, prophylactic molecule, compound, active, or active ingredient. It is contemplated that the agent of the present invention is any antagonist of EphA4-mediated signaling.

In the context of the present invention, an EphA4-mediated signaling antagonist is any agent that results in the complete suppression of, or a substantial decrease in, the levels of EphA4-mediated signaling. Reference herein to “substantial decrease” refers to a decrease of zero to about 90% of the normal level of EphA4-mediated signaling such as a 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 64, 66, 67, 68, 69, 70, 71, 72, 73, 74, 75, 76, 77, 78, 79, 80, 81, 82, 83, 84, 85, 86, 87, 88, 89 or 90% decrease.

Preferably, the EphA4-mediated signaling antagonist of the present invention is a soluble EphA4 receptor or ephrin antagonist or EphA4 receptor or ephrin antagonist, homolog, analog, derivative or structural mimetic. Example antagonists include soluble EphA4 receptor or ligand-binding molecules or mimetics thereof, modified ligand molecules, antibody molecules, small to medium blocking molecules and genetic molecules. Antagonists also include antagonists of kinase activity or levels or other components of the downstream signaling pathway to inhibit EphA4 levels. Any antagonists which act directly or indirectly to antagonize EphA4 mediated-inhibition of axonal growth are contemplated by the present invention. All such molecules are encompassed by the term “agent”.

Reference herein to an “agent” should be understood as a reference to any proteinaceous or non-proteinaceous molecule derived from natural, recombinant or synthetic sources. Useful sources include the screening of naturally produced libraries, chemical molecule libraries as well as combinatorial libraries, phage display libraries and in vitro translation-based libraries.

In one embodiment, the agents of the present invention useful for the complete suppression of, or substantially decreasing, the levels of EphA4-mediated signaling may be chemical or protinaceous molecules.

In relation to proteinaceous molecules, including peptides, polypeptide and proteins, without distinction, the terms mutant, part, derivative, homolog, analog or mimetic are meant to encompass alternative forms of the EphA4-mediated signaling antagonist which completely suppresses or substantially decreases the level of EphA4-mediated signaling.

Mutant forms may be naturally occurring or artificially generated variants of the EphA4-mediated signaling antagonist comprising one or more amino acid substitutions, deletions or additions. Mutants may be induced by mutagenesis or other chemical methods or generated recombinantly or synthetically. Alanine scanning is a useful technique for identifying important amino acids (Wells, Methods Enzymol 202:2699-2705, 1991). In this technique, an amino acid residue is replaced by Alanine and its effect on the peptide's activity is determined. Each of the amino acid residues of the peptide is analyzed in this manner to determine the important regions of the polypeptide. Mutants are tested for their ability to antaganize the EphA4 receptor or its corresponding ephrin and for other qualities such as longevity, binding affinity, dissociation rate, ability to cross membranes or ability to prevent or reduce the amount of gliosis and glial scarring in the nervous system.

Sections of the agents of the present invention encompass EphA4 receptor binding portions or ephrin binding portions of the full-length EphA4-mediated signaling antagonist. Sections are at least 10, preferably at least 20 and more preferably at least 30 contiguous amino acids, which exhibit the requisite activity. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such fragment, irrespective of its means of generation, is to be understood as being encompassed by the term “derivative” as used herein.

Thus derivatives, or the singular derivative, encompass parts, mutants, homologs, fragments, analogues as well as hybrid or fusion molecules and glycosylaton variants. Derivatives also include molecules having a percent amino acid sequence identity over a window of comparison after optimal alignment. Preferably, the percentage similarity between a particular sequence and a reference sequence is at least about 60% or at least about 70% or at least about 80% or at least about 90% or at least about 95% or above such as at least about 96%, 97%, 98%, 99% or greater. Preferably, the percentage similarity between species, functional or structural homologs of the instant agents is at least about 60% or at least about 70% or at least about 80% or at least about 90% or at least about 95% or above such as at least about 96%, 97%, 98%, 99% or greater. Percentage similarities or identities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs. This term also does not exclude modifications of the polypeptide, for example, glycosylations, acetylations, phosphorylations and the like. Included within the definition are, for example, polypeptides containing one or more analogs of an amino acid (including, for example, unnatural amino acids such as those given in Table 3) or polypeptides with substituted linkages. Such polypeptides may need to be able to enter the cell.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids, contemplated herein is shown in Table 3.

TABLE 3
CODES FOR NON-CONVENTIONAL AMINO ACIDS

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Mimetics are another useful group of compounds. The term is intended to refer to a substance which has some chemical similarity to the molecule it mimics, such as, for example, an ephrin, but which antagonizes or agonizes (mimics) its interaction with a target, such as, for example, an EphA4 receptor. A peptide mimetic may be a peptide-containing molecule that mimics elements of protein secondary structure (Johnson et al., Peptide Turn Mimetics in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. Peptide or non-peptide mimetics of an EphA4-mediated signaling antagonist may be useful in the present invention as an agent which decreases levels of EphA4-mediated signaling.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. As described hereinbefore, Alanine scans of peptides are commonly used to refine such peptide motifs. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic. Modelling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, for example, enhance or interfere with the function of a polypeptide in vivo (see, e.g. Hodgson, Bio/Technology 9:19-21, 1991). In one approach, one first determines the three-dimensional structure of a protein of interest by x-ray crystallography, by computer modelling or most typically, by a combination of approaches. Useful information regarding the structure of a polypeptide may also be gained by modelling based on the structure of homologous proteins. An example of rational drug design is the development of HIV protease inhibitors (Erickson et al., Science 249:527-533, 1990).

One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, the formation of complexes between a target or fragment and the agent being tested, or examine the degree to which the formation of a complex between a target or fragment and a known ligand is aided or interfered with by the agent being tested.

The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase. The target may alternatively be expressed as a fusion protein with a tag conveniently chosen to facilite binding and identification.

The present invention also contemplates the use of antibodies and the like for preventing or reducing the amount of gliosis and/or glial scarring and/or inflammation in the nervous system. Suitable agents that may have applicability in the instant invention in this regard include, for example, any protein comprising one or more immunoglobulin domains, and extend to antibodies within the immunoglobulin family of plasma proteins which includes immunoglobulin (Ig)A, IgM, IgG, IgD and IgE. The term “antibody” includes and encompasses fragments of an antibody such as, for example, a diabody, derived from an antibody by proteolytic digestion or by other means including but not limited to chemical cleavage. An antibody may be a “polyclonal antibody” or a “monoclonal antibody”. “Monoclonal antibodies” are antibodies produced by a single clone of antibody-producing cells. Polyclonal antibodies, by contrast, are derived from multiple clones of diverse specificity. The term “antibody” also encompasses hybrid antibodies, fusion antibodies and antigen-binding portions, as well as other antigen-binding proteins such as T-associated binding molecules. In a particularly preferred embodiment the antibodies decrease the level and/or function of an EphA4 receptor, or a molecule required for EphA4 receptor function.

The present invention also extends to genetic agents useful for the complete suppression of, or substantially decreasing, the levels of EphA4-mediated signaling. Suppression includes, but is not limited to, pre- and post-transcriptional gene silencing, post-translational gene silencing, co-suppresion RNAi-mediated gene silencing and methylation. Reference to “RNAi” includes DNA-derived RNAi and synthetic RNAi.

In relation to genetic molecules, the terms mutant, section, derivative, homolog, analog or mimetic have analogous meanings to the meanings ascribed to these forms in relation to proteinaceous molecules. In all cases, variant forms are tested for their ability to function as proposed herein using techniques which are set forth herein or which are selected from techniques which are currently well known in the art.

When in nucleic acid form, a derivative comprises a sequence of nucleotides having at least 60% identity to the parent molecule or portion thereof. A “portion” of a nucleic acid molecule is defined as having a minimal size of at least about 10 nucleotides or preferably about 13 nucleotides or more preferably at least about 20 nucleotides and may have a minimal size of at least about 35 nucleotides. This definition includes all sizes in the range of 10-35 nucleotides including 10, 11, 12, 13, 14, 15, 16, 17, 18, 19, 20, 21, 22, 23, 24, 25, 26, 27, 28, 29, 30, 31, 32, 33, 34 or 35 nucleotides as well as greater than 35 nucleotides including 50, 100, 300, 500, 600 nucleotides or nucleic acid molecules having any number of nucleotides within these values. Having at least about 60% identity means, having optimal alignment, a nucleic acid molecule comprises at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identity with a reference EphA4-mediated signaling antagonist encoding molecule.

The terms “similarity” or “identity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucl Acids Res 25:3389-3402, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The genetic molecules of the present invention are also capable of hybridizing to the genetic agents, or their complement, described herein. Reference herein to “hybridizes” refers to the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low).

Reference herein to a “low stringency” includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as “medium stringency”, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or “high stringency”, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J Mol Biol 5:109-118, 1962). However, the Tₘ of a duplex nucleic acid molecule decreases by 1° C. with every increase of 1% in the number of mismatch base pairs (Bonner and Laskey, Eur J Biochem 46:83-88, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Reference to a “nucleic acid molecule” which modulates the expression of DNA such as, but not limited to, DNA encoding EphA4 and corresponding ephrins, encompasses genetic agents such as DNA (genomic, cDNA), RNA (sense RNAs, antisense RNAs, mRNAs, tRNAs, rRNAs, small interfering RNAs (SiRNAs), micro RNAs (miRNAs), small nucleolar RNAs (SnoRNAs), small nuclear (SnRNAs)) ribozymes, aptamers, DNAzymes or other ribonuclease-type complexes. Other nucleic acid molecules will comprise promoters or enhancers or other regulatory regions which modulate transcription.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts of target genes, such as, but not limited to, genes encoding EphA4 and corresponding ephrins. Expression of such an antisense construct within a cell interferes with target gene transcription and/or translation. Furthermore, co-suppression and mechanisms to induce RNAi or siRNA may also be employed. Alternatively, antisense or sense molecules may be directly administered. In this latter embodiment, the antisense or sense molecules may be formulated in a composition and then administered by any number of means to target cells.

In one embodiment, the present invention employs compounds such as oligonucleotides and similar species for use in modulating the function or effect of nucleic acid molecules such as those encoding a target, i.e. the oligonucleotides induce pre-transcriptional or post-transcriptional gene silencing. This is accomplished by providing oligonucleotides which specifically hybridize with one or more nucleic acid molecules encoding the target gene transcription. The oligonucleotides may be provided directly to a cell or generated within the cell. As used herein, the terms “target nucleic acid” and “nucleic acid molecule encoding a target gene transcript” have been used for convenience to encompass DNA encoding the target, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In one example, the result of such interference with target transcript function is reduced levels of the target. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals.

In the context of the subject invention, the term “oligomeric compound” refers to a polymer or oligomer comprising a plurality of monomeric units. In the context of this invention, the term “oligonucleotide” refers to a nucleic acid oligomer or polymer or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those herein described.

The open reading frame (ORF) or “coding region” which is known in the art to refer to the region between the translation initiation codon and the translation termination codon, is a region which may be effectively targeted. Within the context of the present invention, one region is the intragenic region encompassing the translation initiation or termination codon of the ORF of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns”, which are excised from a transcript before it is translated. The remaining (and, therefore, translated) regions are known as “exons” and are spliced together to form a continuous mRNA sequence. Targeting splice sites, i.e. intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

The efficacy of the agents contemplated by the present invention can be readily determined by, for example, lesioning the central nervous system of an experimental subject, administering an agent to be tested to the lesioned central nervous system for a time and under conditions suitable for assessing the efficacy of said agent, and then, after a period of time, assessing the level of gliosis and/or glial scarring and/or inflammation and/or axonal regeneration at the site of the central nervous system lesion.

Reference herein to “lesioning” means to cut, wound or otherwise induce injury, for example, by using a blade such as a scalpel blade or the application of blunt force.

Reference herein to “experimental subject” includes a subject as hereinafter defined as well as a human which has a lesion to the central nervous system induced by means other than by an experimental means such as disease, condition or accidental injury e.g. car accident.

Reference herein to “assessing” means a reference to qualitative or quantitative assessment.

Accordingly, another aspect of the present invention is a method of determining the efficacy of an agent comprising lesioning the central nervous system of an experimental subject, administering an agent to be tested to the lesioned central nervous system for a time and under conditions suitable for assessing the efficacy of said agent, and then, after a period of time, assessing the level of gliosis and/or glial scarring and/or inflammation and/or axonal regeneration at the site of the central nervous system lesion.

Preferably the central nervous system tissue to be lesioned is the spinal cord.

Accordingly, another aspect of the present invention is a method of determining the efficacy of an agent comprising lesioning the spinal cord of an experimental subject, administering an agent to be tested to the lesioned spinal cord for a time and under conditions suitable for assessing the efficacy of said agent, and then, after a period of time, assessing the level of gliosis and/or glial scarring and/or inflammation and/or axonal regeneration at the site of the spinal cord lesion.

As an example, the efficacy of an agent contemplated by the present invention could be determined by lesioning the spinal cord of an experimental mouse, administering an agent in the form of an EphA4 antagonosit (e.g. ephrinA5-Fc) or an antisense EphA4 oligonucleotide to the lesioned spinal cord for a time and under conditions suitable for assessing the efficacy of said agent, and then, after a period of time, assessing the level of gliosis and/or glial scarring and/or inflammation and/or axonal regeneration at the site of the spinal cord lesion using markers of glial cells and axons.

Agents identified in accordance with the present invention are useful in the treatment of nervous system diseases and injuries characterized by a gliotic response such as gliosis and/or glial scarring and/or inflammation.

Reference herein to “treatment” may mean a reduction in the severity of an existing condition. The term “treatment” is also taken to encompass “prophylactic treatment” to prevent the onset of a condition. The term “treatment” does not necessarily imply that a subject is treated until total recovery. Similarly, “prophylactic treatment” does not necessarily mean that the subject will not eventually contract a condition.

Accordingly, another aspect of the present invention provides a method of preventing or reducing the amount of gliosis and/or glial scarring and/or inflammation in the nervous system of a subject said method comprising administering to said subject an effective amount of an antagonist of EphA4-mediated signaling for a time and under conditions sufficient to prevent or decrease gliosis and/or glial scarring and/or inflammation.

The identification of agents, either genetic or otherwise, capable of modulating EphA4-mediated signaling provides pharmaceutical compositions for use in the therapeutic treatment of gliosis and/or glial scarring and/or inflammation in the nervous system.

Nervous system diseases and injuries contemplated by the present invention include, but are not limited to, traumatic injuries and inflammatory injuries to the brain and spinal cord which result in paralysis.

The agents of the present invention can be combined with one or more pharmaceutically acceptable carriers and/or diluents to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., Remington's Pharmaceutical Sciences, 18^{th} Edition, Mack Publishing Company, Easton, Pa., 1990 (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the modulatory agent of the invention and on its particular physio-chemical characteristics.

Administration of the agent, in the form of a pharmaceutical composition, may be performed by any convenient means known to one skilled in the art. Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, patch and implant.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier, see, e.g., International Patent Publication Number WO 96/11698.

Agents of the present invention, when administered orally, may be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g. Fix, Pharm Res 13:1760-1764, 1996; Samanen et al., J Pharm Pharmacol 48:119-135, 1996; U.S. Pat. No. 5,391,377, describing lipid compositions for oral delivery of therapeutic agents (liposomal delivery is discussed in further detail, infra).

The pharmaceutical forms suitable for injectable use include sterile aqueous solutions (where water-soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the agents in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

For parenteral administration, the agent may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the agents are being administered intrathecally, they may also be dissolved in cerebrospinal fluid.

For transmucosal or transdermal administration, penetrants appropriate to the barrier to be permeated can be used for delivering the agent. Such penetrants are generally known in the art e.g. for transmucosal administration, bile salts and fusidic acid derivatives. In addition, detergents can be used to facilitate permeation. Transmucosal administration can be through nasal sprays or using suppositories e.g. Sayani and Chien, Crit Rev Ther Drug Carrier Syst 13:85-184, 1996. For topical, transdermal administration, the agents are formulated into ointments, creams, salves, powders and gels. Transdermal delivery systems can also include patches.

For inhalation, the agents of the invention can be delivered using any system known in the art, including dry powder aerosols, liquids delivery systems, air jet nebulizers, propellant systems, and the like, see, e.g., Patton, Nat Biotech 16:141-143, 1998; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, for example, air jet nebulizers.

The agents of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (e.g. Putney and Burke, Nat Biotech 16:153-157, 1998).

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's.

In one aspect, the pharmaceutical formulations comprising agents of the present invention are incorporated in lipid monolayers or bilayers such as liposomes, see, e.g., U.S. Pat. Nos. 6,110,490; 6,096,716; 5,283,185 and 5,279,833. The invention also provides formulations in which water-soluble modulatory agents of the invention have been attached to the surface of the monolayer or bilayer. For example, peptides can be attached to hydrazide-PEG-(distearoylphosphatidyl)ethanolamine-containing liposomes (e.g. Zalipsky et al., Bioconjug Chem 6:705-708, 1995). Liposomes or any form of lipid membrane, such as planar lipid membranes or the cell membrane of an intact cell e.g. a red blood cell, can be used. Liposomal formulations can be by any means, including administration intravenously, transdermally (Vutla et al., J Pharm Sci 85:5-8, 1996), transmucosally, or orally. The invention also provides pharmaceutical preparations in which the nucleic acid, peptides and/or polypeptides of the invention are incorporated within micelles and/or liposomes (Suntres and Shek, J Pharm Pharmacol 46:23-28, 1994; Woodle et al., Pharm Res 9:260-265, 1992). Liposomes and liposomal formulations can be prepared according to standard methods and are also well known in the art see, e.g., Remington's; Akimaru et al., Cytokines Mol Ther 1:197-210, 1995; Alving et al., Immunol Rev 145:5-31, 1995; Szoka and Papahadjopoulos, Ann Rev Biophys Bioeng 9:467-508, 1980, U.S. Pat. Nos. 4,235,871, 4,501,728 and 4,837,028.

The pharmaceutical compositions of the invention can be administered in a variety of unit dosage forms depending upon the method of administration. Dosages for typical pharmaceutical compositions are well known to those of skill in the art. Such dosages are typically advisorial in nature and are adjusted depending on the particular therapeutic context, patient tolerance, etc. The amount of agent adequate to accomplish this is defined as the “effective amount”. The dosage schedule and effective amounts for this use, i.e., the “dosing regimen” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., Remington's; Egleton and Davis, Peptides 18:1431-1439, 1997; Langer, Science 249:1527-1533, 1990.

In accordance with these methods, the agents and/or pharmaceutical compositions defined in accordance with the present invention may be co-administered in combination with one or more other agents. Reference herein to “co-administered” means simultaneous administration in the same formulation or in two different formulations via the same or different routes or sequential administration by the same or different routes. Reference herein to “sequential” administration is meant a time difference of from seconds, minutes, hours or days between the administration of the two types of agents and/or pharmaceutical compositions. Co-administration of the agents and/or pharmaceutical compositions may occur in any order. Agents which are particularly preferred in this regard are agents which promote neurogenesis and/or axon growth and/or inhibit inflammation such as, but not limited to cytokines and growth factors (e.g. LIF, growth hormone etc) and inhibitors of inflammatory cytokines such as INFγ antagonists.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering the agents directly, they could be produced in the target cell, e.g. in a viral vector such as described above or in a cell based delivery system such as described in U.S. Pat. No. 5,550,050 and International Patent Publication Numbers WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application Number 0 425 731A and International Patent Publication Number WO 90/07936.

In yet another aspect, the present invention provides kits comprising the compositions e.g. agents of the present invention. The kits can also contain instructional material teaching the methodologies and uses of the invention, as described herein.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features. It is also to be understood that unless stated otherwise, the subject invention is not limited to specific formulation components, manufacturing methods, dosage regimes, or the like, as such may vary.

Combination therapy is another aspect of the present invention. Combination therapy includes the simultaneous or sequential administration of, in any order, an EphA4 antagonist and another agent such as an antagonist of an inflammatory cytokine or a blocker of EphA4-neurite interaction. Examples of agents include antibodies (native, single chain, chimeric or recombinant or fragments thereof) as well as a range of small molecule therapeutics and cytokines such as LIF or growth hormone receptor agonists.

The present invention is further described by the following non-limiting examples.

### EXAMPLE 1

### Materials and Methods

The following materials and methods are used in the subsequent Examples which follow.

Mice

Adult EphA4−/− and C57BL/6 mice, 3-12 months old and maintained as previously described (Coonan et al., J Comp Neurol 436:248-262, 2001), were used in this study.

Spinal Cord Lesions

Mice were anesthetized with a mixture of ketamine and xylazine (100 mg/kg and 16 mg/kg, respectively). The spinal cord was exposed via a laminectomy, in which 2-3 vertebral arches were removed at levels T12-L1, corresponding to the level of the lumbar enlargement. A spinal left hemisection at T12 was performed using a fine corneal blade (cut twice in the same place to ensure complete section) and the overlying muscle and skin were then sutured. Hemisection was performed on 44 wildtype and 37 EphA4−/− mice. Of these, 28 wildtype and 19 EphA4−/− mice were used for immunohistochemical studies; the remaining animals were behaviorally assessed and the extent of regeneration subsequently examined by axonal tracing.

Anterograde Tracing

Five weeks after spinal cord lesion, tetramethylrhodamine dextran (“Fluoro-Ruby”, MW 10,000 kD) was injected into the spinal cord at the level of the cervical enlargement, ipsilateral to the lesion, via a glass pipette attached to a Hamilton syringe. After a further 7-day survival period, the animals were perfused with 4% paraformaldehyde. Longitudinal serial sections of spinal cord were cut at 50 μm on a freezing microtome and sections were mounted on gelatinized slides and examined using fluorescence and confocal microscopy.

This technique labeled all descending axonal pathways ipsilateral to the injection site but none contralateral to the injection site.

The number of labeled axons running rostrally to caudally in the white matter of all intact serial sections (8-10 per spinal cord) was counted at ×400, with the aid of a grid and by focusing up and down through the sections at 2.5 mm and 50-100 μm proximal to the lesion site and 50-100 μm, 1 mm and 5 mm distal to the hemisection. The lumbar site of the lesion precluded analysis of regrowth longer than 5 mm due to termination of the fibers and commencement of the Cauda Equina. Significance of results was analyzed using the Student's t-test.

Retrograde Tracing

The lumbar spinal cord below the lesion was exposed via a lower lumbar laminectomy. Fast Blue (2% (w/v), 0.3 μl per injection; EMS-POLYLOY GmBH,Groβ-Umstadt, Germany), which labels the neuronal soma of axons damaged by the injection, was injected into the spinal cord ipsilateral to the lesion site with a glass micropipette attached to a Hamilton syringe. After a 5-day survival period, the animals were perfused with 4% paraformaldehyde in PBS. The brain and spinal cord were removed, post-fixed for 24 hours in 20% sucrose in fixative before being serially sectioned at 50 μm on a freezing microtome in the coronal/transverse plane. Injections were considered successful by confirmation of a unilateral injection site in the operated spinal cord longitudinal sections. Qualitative and quantitative comparisons of labeled neurons were made by mapping the locations of labeled cells in every fourth section of a series using a computer-linked digitizing system (MD3 microscope digitizer and MD-plot software; Minnesota Datametrics Corporation, MN, USA).

Behavioral Analysis

Stride length: Prior to and following hemisection, mice were foot-printed by painting their hind paws with non-toxic ink and placing them in a tunnel on blotting paper (wildtype, n=7 and EphA4−/− n=9 mice). Stride length was determined by measurement of multiple successive steps and results were expressed as a percentage of each animal's own baseline stride length.

Grid walking: The ability of wildtype (n=5) and EphA4−/− (n=7) mice to walk on a horizontal or angled (75° from horizontal) wire grid (1.2×1.2 cm grid spaces, 35×45 cm total area) was determined in order to assess their locomotion (Ma et al., Exp Neurol 169:239-254, 2001). The mice were tested 1, 2 and 3 months after the spinal cord hemisection and compared with non-lesioned mice from each group. On the horizontal grid each mouse was allowed to walk freely around the grid for 5 min, during which a minimum 2 min of walking time was required. On the angled grid, each mouse was measured over 10 climbs. If the left hind-paw protruded entirely through the grid, with all toes and heel extended below the wire surface, it was counted as a misstep. The total number of steps taken with the left hind limb was also counted. The results were expressed as the percentage of accurate foot steps and significance was analyzed using the Student's t-test.

Sensory and motor ability-grasp test: The ability of hemisected and non-lesioned wildtype (n=5) and EphA4−/− (n=7) mice to grasp a 7 mm diameter rod was tested on the left hindlimb. The hindlimbs of the mice were lifted 2 cm from the table top while allowing the forelimbs to remain in contact with the table. Grasp ability was tested by lightly touching the left foot pad with the rod and assessing the response based on a scale from 0-4: 0, no movement of paw and toes; 1, partial movement of the paw, no movement of the toes; 2 partial grasp, slight movement of toes and paw; 3, weak full grasp, not maintained with gentle rod movement; 4, strong grasp, maintained with gentle rod movement. Mice were graded at least 3 times in parallel with the grid tests described. Results were expressed as the mean±SEM of each group's score and significance was analyzed using the Student's t-test.

Immunohistochemistry and Astrocyte Counts

Standard immunohistochemical procedures, using rabbit anti-GFAP (1:500, Dako), mouse anti-CSPG (1:200, Sigma) and rabbit anti-EphA4 were followed. The rabbit anti-EphA4 antibody (available from the inventors) was prepared against a peptide corresponding to amino acids 938-953 of the intracellular SAM domain of EphA4 (Genbank accession number NM007936) using standard procedures (Cooper and Paterson, Current protocols in molecular biology, eds Ausubel et al. 11.12.11-11.12.19, John Wiley & Sons, New York, 2000). The number of hypertrophic astrocytes, as well as the total number of GFAP-expressing astrocytes, were counted in a 0.25 mm² grid at and 2.5 mm proximal to the lesion site, in every third serial longitudinal 8 μm section. Hypertrophic astrocytes were defined as intensely stained GFAP-positive cells with a large cell body and multiple thick long processes. Non-hypertrophic astrocytes stained less intensely for GFAP and had a small cell body with thin, less complex processes. Hypertrophic astrocytes were more than twice the size of non-hypertrophic astrocytes.

Astrocyte and Neuronal Cultures and Neurite Length Measurement

Purified astrocyte and neuronal cultures were prepared as previously described (Turnley et al., Nat Neurosci 5:1155-1162, 2002). For analysis of neurite length, E16 cortical neurons were plated at 5,000/well in chamber slides (Falcon, USA) containing wildtype or EphA4−/− astrocyte monolayers or which were poly-DL-ornithine/laminin coated. In some experiments, astrocytes were pretreated for 1 hr with monomeric EphrinA5-Fc (0.15, 1.5, 10 μg/ml) or complexed EphrinA5-Fc (1.5 μg/ml complexed with 0.15 μg/ml anti-human IgG (Vector) for 30 min at room temperature prior to addition). After 22 hrs, cells were fixed and immunostained for the neuronal marker βIII-tubulin (1:2000, Promega). Neurite length was measured using image analysis as previously (Turnley et al., Neuroreport 9:1987-1990, 1998). Significance of differences in the mean neurite lengths was analyzed using the Student's t-test.

For biochemical analysis of astrocytes, factors as indicated were added to 80% confluent monolayers in 10 cm plates (Falcon, USA) for the times indicated. EphrinA5-Fc (available from the inventors) was pre-complexed as above.

Immunoprecipitation and Western Analysis

Cells were lysed and a sample kept aside for analysis of total protein levels. The remainder of the lysate was used for immunoprecipitation in EphA4 or Rho activation assays. EphA4 activation was determined by immunoprecipitation of phosphorylated proteins using anti-phosphotyrosine (Cell Signaling), followed by Western transfer and detection of activated or total EphA4 using a rabbit anti-EphA4 antibody (kindly provided by Dr. D. Wilkinson, National Institute for Medical Research, London). Rho activation assays were performed using the Rhotekin RBD assay, according to the manufacturer's instructions (Upstate, USA). Total EphA4 and β-actin expression levels were determined in non-lesioned and 7 d post-lesioned spinal cords by Western analysis using rabbit anti-EphA4 antibody as above and mouse anti-β-actin antibody (Sigma). Densitometry was performed on the autoradiographs using NIH Image software to determine relative levels of the EphA4 bands and normalized to β-actin levels.

Cell Proliferation Assay

The [3-(4,5-dimethylthiazol-2-yl)-2,5-diphenyl]tetrazolium bromide (MTT) assay, which determines mitochondrial activity in living cells, is commonly used as a proliferation assay (Mosmann, J Immunol Methods 65:55-63, 1983). Living cells transform the tetrazolium ring into dark blue formazan crystals, which can be quantified by reading the optical density (O.D.); an increase in O.D. correlates with an increase in cell number over time. Wildtype and EphA4−/− astrocytes were plated on 96 well plates (Falcon) at 3×10³ cells/well in DMEM supplemented with 10% FCS in the presence or absence of either LIF (1000 U/ml) or IFN-γ (100 U/ml). The MTT assay was performed at 2, 24, 48 and 72 hours after plating. MTT (0.25 mg/ml) was incubated with the cells at each timepoint for 2 hours at 37° C., the cells were then lysed with an equal volume of acidic isopropanol (0.04M HCL in absolute isopropanol) and the O.D. of the formazan product was measured at 550-650 nm.

EphrinA5-Fc Injection

Injections of PBS/EphrinA5-Fc (0.687 mg/injection) or PBS alone were made I.P. starting 2 hours post surgery and then every 24 hours for up to 2 weeks.

### EXAMPLE 2

### Tracing of Lesioned Axons Indicates Extensive Regeneration by 6 Weeks

As EphA4−/− mice have some developmental corticospinal tract abnormalities, with some axons terminating prematurely or aberrantly crossing the midline (Dottori et al., Proc Natl Acad Sci USA 95:13248-13253, 1998; Coonan et al., J Comp Neurol 436:248-262, 2001), this precluded the use of standard corticospinal tract tracing techniques. In addition, we did not wish to make assumptions about effects of the EphA4 deletion on other axonal tracts. We therefore chose to use an anterograde tracing technique, whereby the tracer was injected into the cervical spinal cord, well above the lumbar lesion site. This allowed us to assess general regeneration of individual axons. Use of this technique in unlesioned wildtype and EphA4−/− mice showed equivalent labeling of descending axonal pathways ipsilateral to the injection site but none contralateral to the injection site.

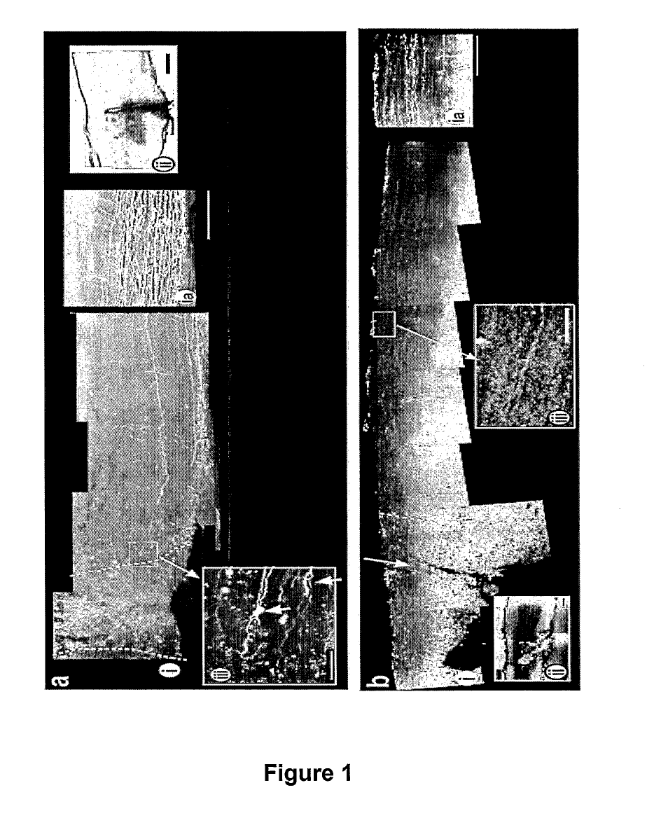

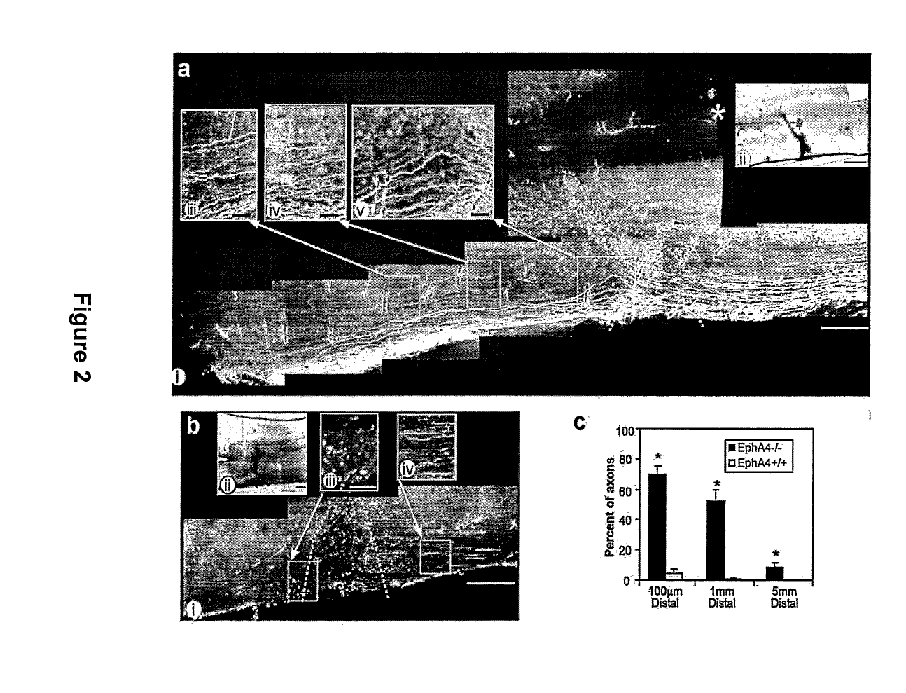

At 6 days post-lesion, in both wildtype and EphA4−/− mice, anterograde labeling revealed no labeled fibers within the lesion site, although there were axons with growth cones near the lesion site in the EphA4−/− mice (FIG. 1a, b). By six weeks after spinal hemisection, however, many anterogradely labeled axons crossed the EphA4−/− lesion site (FIG. 2a; Supplementary FIG. 2a-d), unlike in the wildtype lesion (FIG. 2b). Only sections in which the entire length of spinal cord was intact were included in the results and axons that were close to the pial surface were excluded from the counts. Of the anterogradely labeled axons that reached the lesion site (70.8±14.7 in EphA4−/− mice compared with 37.25±9.6 in wildtype), 70% of EphA4−/− axons crossed it (as measured 100 μm distally) and, of these, 75% were maintained at 1 mm and 15% at 5 mm distal to the injury (FIG. 2a, c). By contrast, in wildtype mice approximately 4% of the fibers crossed the lesion site and virtually none of these were detected at 1 mm and 5 mm distal. Since considerably more axons in the EphA4−/− mice reached the lesion site, the magnitude of the difference is even more pronounced and demonstrates that there is considerable inhibition of regrowth of wildtype axons upstream of the lesion. Although the regenerating axons that crossed the lesion site at 6 weeks appeared “wavy” (FIG. 2av), as is typical during regeneration, the vast majority could be traced as running in an unhindered rostral to caudal line (FIG. 2a). While some fibers showed branching or deviation, particularly after the lesion site (FIG. 2aiii, aiv), a montage of confocal micrographs covering the entire lesion area and across the midline (FIG. 2a) revealed that no fibers crossed from the unlesioned side and contributed to the labeled fiber bundle running through or distal to the lesion. Thus, the large number of labeled axons passing through and beyond the lesion site in EphA4−/− mice can only be attributed to genuine regrowth of severed axons.

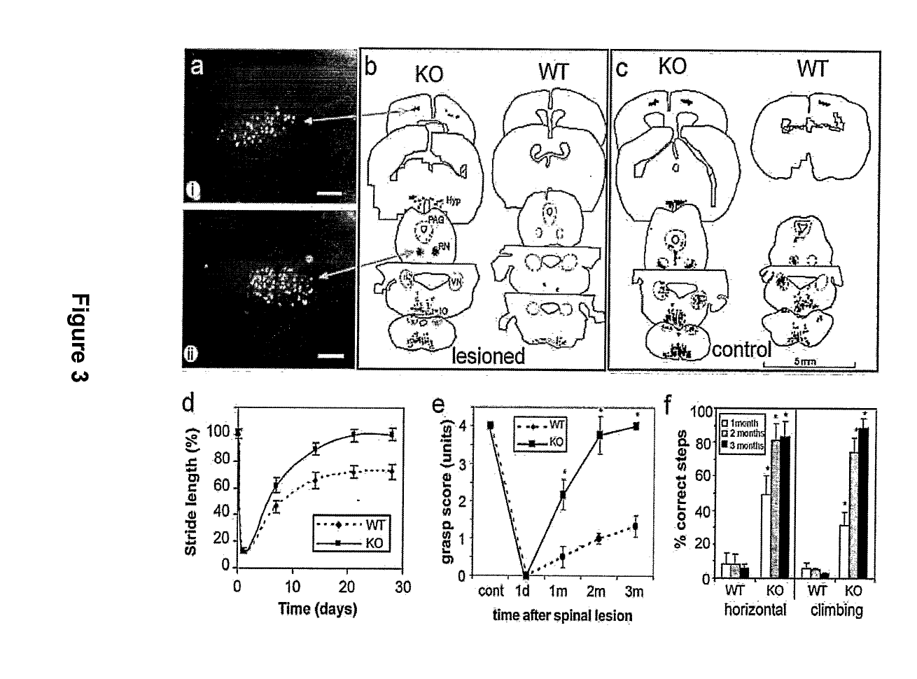

As the anterograde tracing used in this study labeled all the descending spinal pathways, retrograde tracing was used to identify which specific axonal tracts had regenerated. This revealed that in the EphA4−/− but not the wildtype mice, multiple axonal tracts showed regeneration. Labeled neurons were present in motor cortex (corticospinal tract) and the red nucleus (rubrospinal tract), as well as in the hypothalamus, the vestibular and reticular nuclei and the periaqueductal grey matter (FIG. 3a, b), the same regions that were labeled in the non-lesioned control EphA4−/− and wildtype mice (FIG. 3c). In the wildtype mice, only a small number of bilaterally projecting reticulospinal neurons were labeled following lesion.

### EXAMPLE 3

### Functional Recovery of EphA4−/− Mice

The axonal regeneration observed in EphA4−/− mice also had a functional correlate. Mice were behaviorally assessed, first by measuring their stride length (Bregman et al., Nature 378:498-501, 1995) prior to and from 24 hrs to 4 weeks following spinal hemisection. At 24 hrs both EphA4−/− and wildtype mice showed minimal function. EphA4−/− mice regained 100% of their baseline stride length within 3 weeks, while wildtype mice showed only 70% recovery (FIG. 3d) and did not improve thereafter. In addition, 1 month following hemisection, the ipsilateral hindpaw grip strength (FIG. 3e) and ability to walk on a grid (FIG. 3f) were dramatically improved in EphA4−/− mice compared with wildtype. These functions continued to improve up to 3 months post-lesion. Non-lesioned EphA4−/− and wildtype mice both achieved maximal scores in these tests.

### EXAMPLE 4

### Lack of Astrocytic Gliosis in EphA4−/− Mice

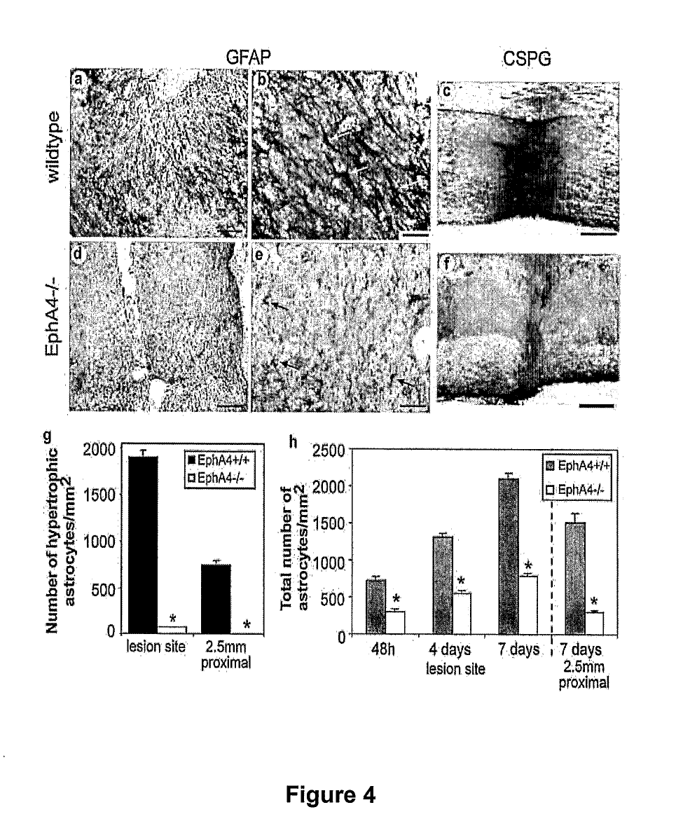

A striking feature of the hemisected EphA4−/− spinal cord was the virtual absence of astrocytic gliosis, as assessed by GFAP expression, compared with the wildtype (FIG. 4a, b, d, e). At day 7, the vast majority (90.4%) of the GFAP-positive astrocytes at the wildtype lesion site were hypertrophic and stained very strongly for GFAP, whereas only 7.4% of EphA4−/− astrocytes were hypertrophic (FIG. 4g). Overall, the total number of GFAP-positive cells was fewer at the EphA4−/− hemisection over the first 7 days post-lesion, and this was strikingly the case proximal to the lesion site (FIG. 4h). In non-lesioned cases there was no difference in astrocyte numbers between EphA4−/− and wildtype mice (wildtype 836.8±108.3/mm² compared to EphA4−/− 825.6±98.1/mm²). The lack of glial response resulted in a marked reduction in the size of the glial scar of EphA4−/− mice at 6 weeks post-lesion as assessed by immunostaining for a component of the glial scar, CSPG (FIG. 4c, f).

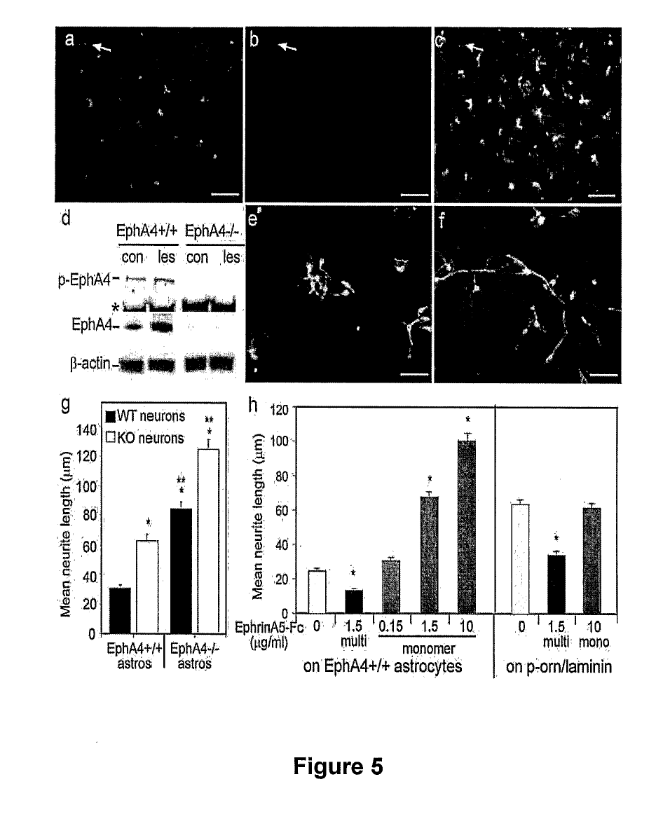

Since EphA4 expression appeared to regulate both the level of regeneration and gliosis following lesioning, we next examined whether EphA4 expression was upregulated following spinal hemisection. Immunostaining and Western analysis (FIG. 5a, d) revealed that EphA4 expression occurred at very low levels, undetectable by immunostaining in non-lesioned animals except on some motor neurons (Supplementary FIG. 3b-d). However, expression and phosphorylation were upregulated following spinal lesion (FIG. 5d), and almost exclusively on GFAP-expressing astrocytes at the lesion site (FIG. 5a-c). Low levels of EphA4 were found on anterogradely labeled axons proximal to the lesion site (Supplementary FIG. 3e-g). A ligand for EphA4, EphrinB3, was also expressed on regenerating axons, as well as on some astrocytes.

### EXAMPLE 5

### EphA4 Expression on Astrocytes Inhibits Neurite Outgrowth

The expression of EphA4 on astrocytes was investigated as to whether this inhibits neurite outgrowth of cortical neurons in vitro. E16 cortical neurons were plated onto monolayers of either wildtype or EphA4−/− astrocytes and the length of the longest neurite was measured 22 hours later. This revealed a 2-3 fold increase in outgrowth on EphA4−/− astrocytes compared with wildtype astrocytes. (FIG. 5e-g). This effect appeared to be directly due to expression of EphA4 on the astrocytes, as similar results were obtained when neurons were grown on 293T cells transfected with EphA4 (neurite length on non-transfected 293T cells was 80.4±3.3 μm compared with 30.2±1.9 μm on EphA4-transfected cells). The increased neurite outgrowth of EphA4−/− neurons compared with wildtype neurons, on both wildtype and EphA4−/− astrocytes (FIG. 5g), suggests that EphA4 expressed on the neurons may also inhibit neurite outgrowth, as has been previously suggested (Wahl et al., J Cell Biol 149:263-270, 2000; Kullander et al., Genes Dev 15:877-888, 2001), and may contribute to the regeneration observed in EphA4−/− mice. Inhibition of neurite outgrowth on astrocytes was potently blocked in a dose-dependent manner by the addition of monomeric EphrinA5-Fc, which strongly binds to EphA4 in the astrocyte monolayer; however, it had no effect on neurons grown on laminin-coated glass slides (FIG. 5h). Conversely, addition of complexed EphrinA5-Fc inhibited neurite outgrowth on glass slides, as previously described (Wahl et al., J Cell Biol 149:263-270, 2000; Kullander et al., Genes Dev 15:877-888, 2001), and further inhibited outgrowth on astrocytes (FIG. 5h). This indicates that blocking of EphA4 on astrocytes, but not on neurons, enhances neurite outgrowth, whereas activation of EphA4 on both neurons and astrocytes inhibits neurite outgrowth. Both results point directly to the activation of EphA4 by a ligand as being the mechanism for neurite inhibition. In vivo, a possible activator of the neurite responses to EphA4 expression on the astrocytes was EphrinB3, which has been shown to transduce signals (Palmer et al., Mol Cell 9:725-737, 2002) and which was expressed by regenerating axons in the spinal cord.

### EXAMPLE 6

### Rho Activation and Proliferation is Decreased in EphA4−/− Astrocytes

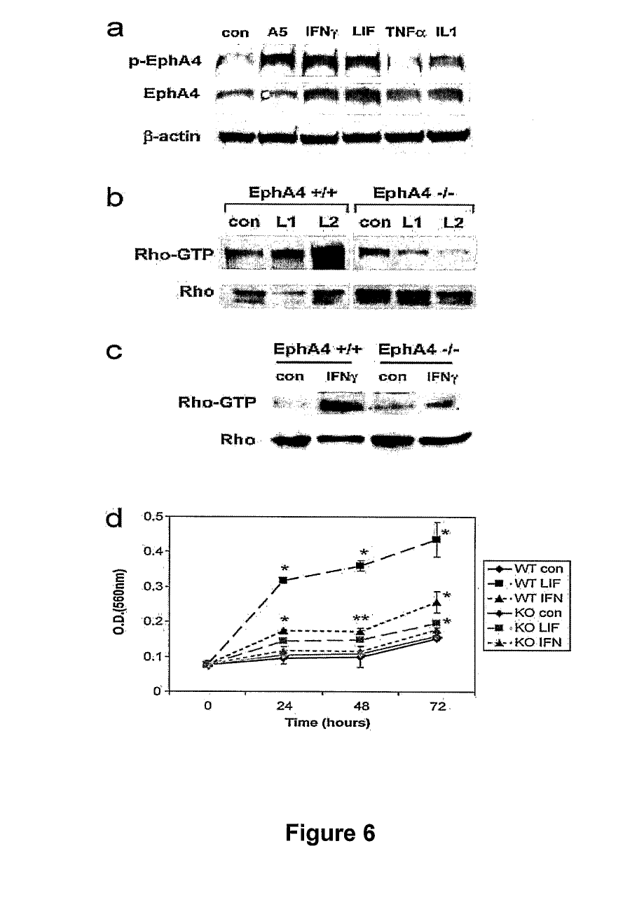

Given that previous studies have demonstrated that gliosis is mediated by inflammatory cytokines, including interferon-γ (IFNγ) and leukemia inhibitory factor (LIF) (Yong et al., Proc Natl Acad Sci USA 88:7016-7020, 1991; Balasingam et al., J Neurosci 14:846-856, 1994; Sugiura et al., Eur J Neurosci 12:457-466, 2000) we then investigated whether these cytokines play a role in the upregulation of EphA4 on astrocytes. IFNγ and LIF upregulated EphA4 expression by 56% and 69% respectively, whereas interleukin-1 (Il-1) and tumor necrosis factor-α (TNFα) had no effect (FIG. 6a). In order to directly address the question of whether EphA4 expression is accompanied by downstream activation which could lead to astrocytic responses, we examined whether EphA4 is phosphorylated. Both IFNγ and LIF upregulated EphA4 phosphorylation 2 fold, in a similar manner to the addition of a soluble multimeric EphA4 ligand, EphrinA5-Fc (FIG. 6a). In addition, this led to a marked increase in activation of the small GTPase, Rho, a major regulator of cytoskeletal changes (Hall, Science 279:509-514, 1998) downstream of Eph receptor signaling (Wahl et al., J Cell Biol 149:263-270, 2000; Shamah et al., Cell 105:233-244, 2001). Increased Rho activation occurred both in wildtype spinal cord tissue removed from the lesion site (FIG. 6b) and in cultured astrocytes (FIG. 6c); no such response was observed using cells or tissue removed from lesioned EphA4−/− animals. Activation of Rho in astrocytes, as well as in neurons and oligodendrocytes, has also recently been reported in spinal cord following injury (Dubreuil et al., J Cell Biol 162:233-243, 2003).

### EXAMPLE 7

### Injection of EphrinA5-Fc Increases Axon Regeneration

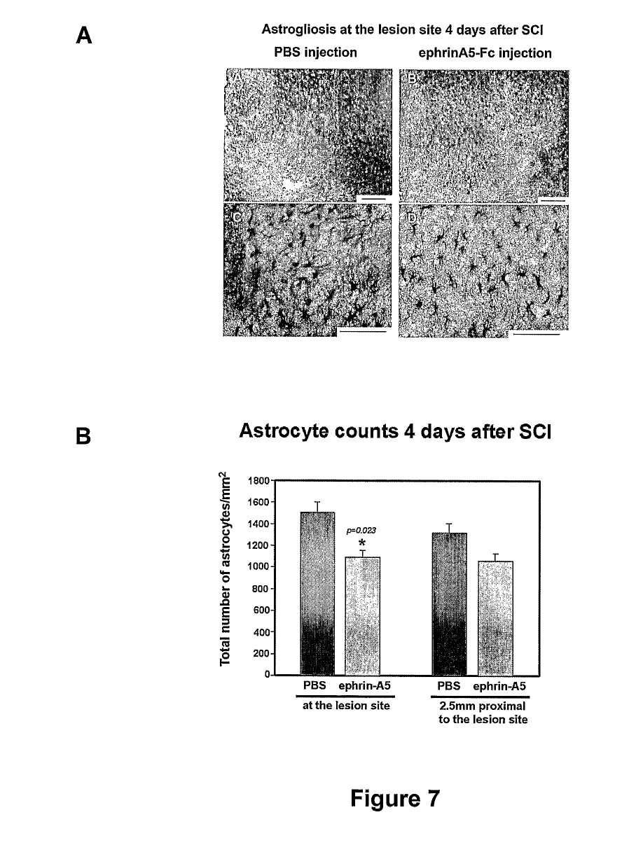

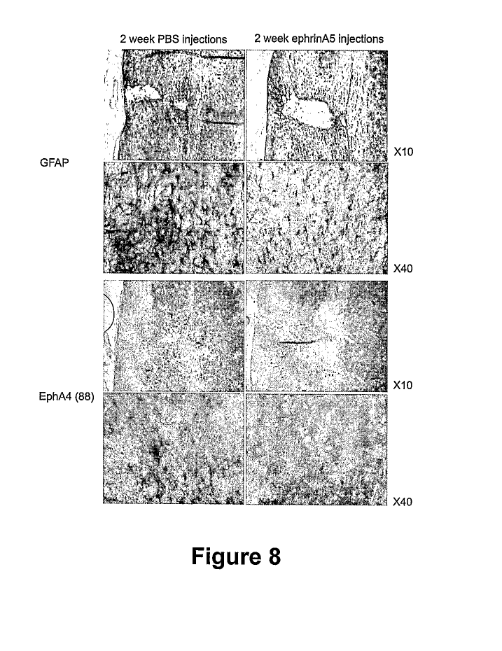

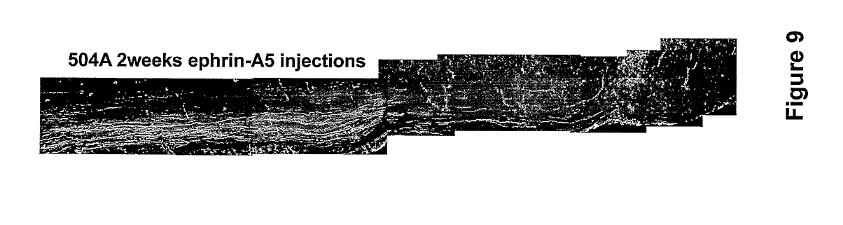

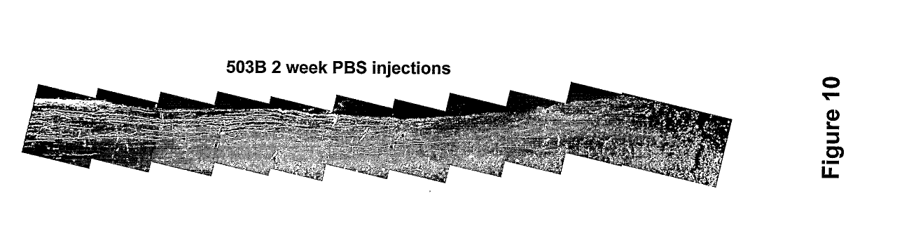

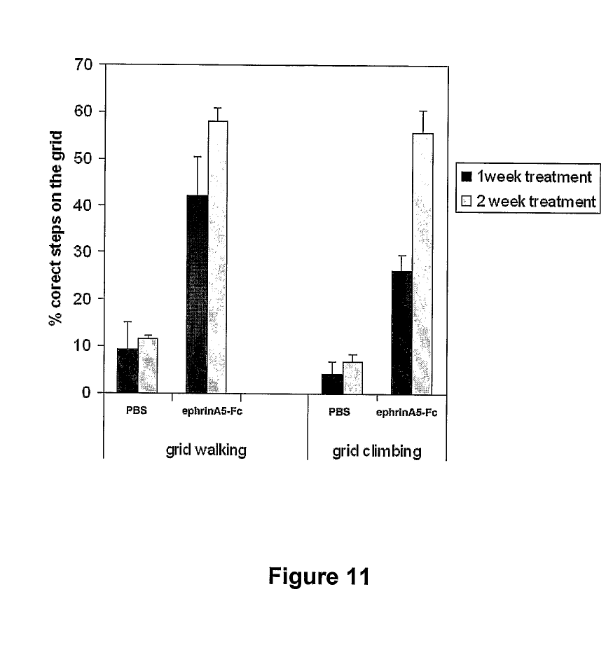

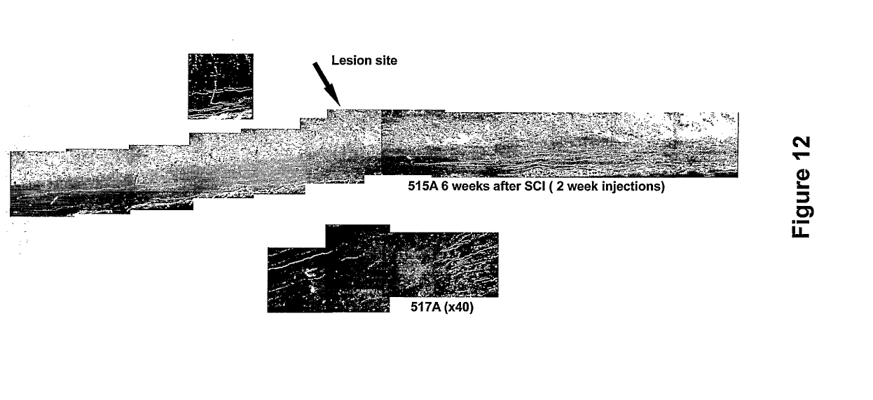

Monomeric ehrinA5-Fc blocks activation of EphA4 in vitro. Following ephrinA5-Fc injection, astrogliosis in mice that have undergone a spinal lesion is significantly reduced compared to those that have been injected with PBS alone (FIG. 7A and 7B). Similarly, ephrinA5-Fc injection for 2 weeks also inhibits EphA4 up-regulation in the injured spinal cord (FIG. 8). When axonal regeneration is examined in the spinal cords of mice 2 weeks after lesioning, significant collateral sprouting and regeneration near the lesion site occurs following ephrinA5-Fc injection (FIG. 9) when compared to PBS alone (FIG. 10). This regeneration is reflected in a significant improvement in grid walking and climbing in mice that have undergone SCI and ephrinA5-Fc injection (FIG. 11). Six weeks post SCI, significant axon regeneration across the lesion site is observed in mice that have been injected with ephrinA5-Fc (FIG. 12).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any to or more of said steps or features.

### BIBLIOGRAPHY

Akimaru et al., Cytokines Mol Ther 1:197-210, 1995
Altschul et al., Nucl Acids Res 25:3389-3402, 1997
Alving et al., Immunol Rev 145:5-31, 1995
Balasingam et al, J Neurosci 14:846-856, 1994
Bandtlow and Schwab, Glia 29:175-181, 2000
Bergemann et al., Oncogene 16:471-480, 1998
Bonner and Laskey, Eur J Biochem 46:83-88, 1974
Bregman et al., Nature 378:498-501, 1995
Bruckner et al., Science 275:1640-1643, 1997
Chen et al., Nature 403:434-439, 2000
Coonan et al., J Comp Neurol 436:248-262, 2001
Cooper and Paterson, Current protocols in molecular biology, eds Ausubel et al. 11.12.11-11.12.19, John Wiley & Sons, New York, 2000
Connor et al., Dev Biol 193:21-35, 1998
Davy et al., Genes Dev 13:3125-3135, 1999
Dottori et al., Proc Natl Acad Sci USA 95:13248-13253, 1998
Dou et al., J Neurosci 14:7616-7628, 1994
Dubreuil et al., J Cell Biol 162:233-243, 2003
Egleton and Davis, Peptides 18:1431-1439, 1997
Eph Nomenclature Committee, Cell 90:403-404, 1997
Erickson et al., Science 249:527-533, 1990
Fix, Pharm Res 13:1760-1764, 1996
Flanagan and Vanderhaeghen, Ann Rev Neurosci 21:309-345, 1998
Gale et al., Oncogene 13:1343-1352, 1996
Hall, Science 279:509-514, 1998
Hodgson, Bio/Technology 9:19-21, 1991
Holder and Klein, Development 126:2033-2044, 1999
Holland et al., Nature 383:722-725, 1996
Imondi et al., Development 127:1397-1410, 2000
Iwamasa et al., Dev Growth Diff 41:685-698, 1999
Johnson et al., Peptide Turn Mimetics in Biotechnology and Pharmacy, Pezzuto et al., Eds., Chapman and Hall, New York, 1993
Kalo and Pasquale, Cell Tissue Res 298:1-9, 1999
Kilpatrick et al., Mol Cell Neurosci 7:62-74, 1996
Kim et al., Neuron 38:187-199, 2003
Kullander et al., Genes Dev 15:877-888, 2001
Kury et al., Mol Cell Neurosci 15:123-140, 2000
Langer, Science 249:1527-1533, 1990
Ma et al., Exp Neurol 169:239-254, 2001
Marmur and Doty, J Mol Biol 5:109-118, 1962
Martone et al., Brain Res 771:238-250, 1997
McKeon et al., J Neurosci 11:3398-3411, 1991
McKerracher et al., Neuron 13:805-811, 1994
Mori et al., Brain Res Mol Brain Res 29:325-335, 1995
Mosmann, J Immunol Methods 65:55-63, 1983
Mueller, Ann Rev Neurosci 22:351-388, 1999
Mukhopadhyay et al., Neuron 13:757-767, 1994
Nakanioto, Int J Biochem Cell Biol 32:7-12, 2000
Niederost et al., J Neurosci 19:8979-8989, 1999
O'Leary and Wilkinson, Curr Opin Neurobiol 9:65-73, 1999
Palmer et al., Mol Cell 9:725-737, 2002
Patton, Nat Biotech 16:141-143, 1998
Putney and Burke, Nat Biotech 16:153-157, 1998
Remington's Pharmaceutical Sciences, 18^{th} Edition, Mack Publishing Company, Easton, Pa., 1990
Samanen et al., J Pharm Pharmacol 48:119-135, 1996
Sayani and Chien, Crit Rev Ther Drug Carrier Syst 13:85-184, 1996
Shamah et al., Cell 105:233-244, 2001
Simonen et al., Neuron 38:201-211, 2003
Stichel et al., Eur J Neurosci 7:401-411, 1995
Stichel and Muller, Cell Tissue Res 294:1-9, 1998
Stichel et al., Eur J Neurosci 11:632-646, 1999
Sugiura et al., Eur J Neurosci 12:457-466, 2000
Suntres and Shek, J Pharm Pharmacol 46:23-28, 1994
Szoka and Papahadjopoulos, Ann Rev Biophys Bioeng 9:467-508
Turnley et al., Neuroreport 9:1987-1990, 1998
Turnley et al., Nat Neurosci 5:1155-1162, 2002
Vutla et al., J Pharm Sci 85:5-8, 1996
Wahl et al., J Cell Biol 149:263-270, 2000
Wells, Methods Enzymol 202:2699-2705, 1991
Woodle et al., Pharm Res 9:260-265, 1992
Yong et al., Proc Natl Acad Sci USA 88:7016-7020, 1991
Zalipsky et al., Bioconjug Chem 6:705-708, 1995
Zheng et al., Neuron 38:187-199, 2003

1. A method of preventing or reducing the amount of gliosis and/or glial scarring and/or inflammation and/or inhibition of axonal growth in the nervous system of a subject said method comprising administering to said subject an agent which decreases the level and/or function of an Eph receptor, or a molecule required for Eph receptor function, in order to decrease levels of Eph receptor-mediated signaling.

2. The method of claim 1 wherein the Eph receptor is the EphA4 receptor or a homolog, paralog, ortholog, derivative or functional equivalent thereof.

3. The method of claim 2 wherein the Eph receptor is the EphA4 receptor.

4. The method of claim 1 wherein the agent is a proteinaceous or non-proteinaceous molecule.

5. The method of claim 4 wherein the agent is a EphA4 receptor antagonist, homolog, analog, derivative or structural mimetic.

6. The method of claim 4 wherein the agent is an ephrin antagonist, homolog, analog, derivative or structural mimetic.

7. The method of claim 4 wherein the agent is a nucleic acid molecule.

8. The method of claim 7 wherein the nucleic acid molecule is an anti-sense, sense, DNA-derived RNAi or synthetic RNAi directed to the EphA4 receptor mRNA transcript.

9. The method of claim 4 wherein the agent is an antibody or a derivative, recombinant, chimeric or deimmunized form thereof.

10. The method of claim 1 wherein the nervous system is the central nervous system (CNS).

11. The method of claim 1 wherein the subject is a human.

12. A method of determining the efficacy of an agent comprising lesioning the central nervous system of an experimental subject, administering an agent to be tested to the lesioned central nervous system for a time and under conditions suitable for assessing the efficacy of said agent, and then, after a period of time, assessing the level of gliosis and/or glial scarring and/or inflammation and/or axonal growth regeneration at the site of the central nervous system lesion.

13. The method of claim 12 wherein the central nervous system to be lesioned out is spinal cord.

14. The method of claim 12 wherein the agent inhibits an Eph receptor or Eph receptor-mediated signaling.

15. The method of claim 14 wherein the Eph receptor is EphA4 or a homolog, paralog, ortholog, derivative or functional equivalent thereof.

16. The method of claim 15 wherein the Eph receptor is the EphA4 receptor.

17. The method of claim 12 wherein the agent is a proteinaceous or non-proteinaceous molecule.

18. The method of claim 17 wherein the agent is an EphA4 antagonist, homolog, analog, derivative or structural mimetic.

19. The method of claim 17 wherein the agent is an ephrin antagonist, homolog, analog, derivative or structural mimetic.

20. The method of claim 17 wherein the agent is a nucleic acid molecule.

21. The method of claim 20 wherein the nucleic acid molecule is an anti-sense, sense, DNA-derived RNAi or synthetic RNAi directed to the EphA4 receptor mRNA transcript.

22. The method of claim 17 wherein the agent is an antibody or a derivative, recombinant, chimeric or deimmunized form thereof.

23. The method of claim 12 wherein the nervous system is the central nervous system (CNS).

24. The method of claim 12 wherein the subject is a human.

25. A method of determining the efficacy of an agent comprising contacting a cell with an agent to be tested in vitro for a time and under conditions suitable for assessing the efficacy of said agent, and then, after a period of time, assessing the propensity of the cell to be involved in gliosis and/or glial scarring and/or inflammation and/or axonal regeneration.

26. The method of claim 25 wherein the agent inhibits an Eph receptor or Eph receptor-mediated signaling.

27. The method of claim 25 wherein the Eph receptor is EphA4 or a homolog, paralog, ortholog, derivative or functional equivalent thereof.

28. The method of claim 27 wherein the Eph receptor is the EphA4 receptor.

29. The method of claim 25 wherein the agent is a proteinaceous or non-proteinaceous molecule.

30. The method of claim 29 wherein the agent is an EphA4 antagonist, homolog, analog, derivative or structural mimetic.

31. The method of claim 29 wherein the agent is an ephrin antagonist, homolog, analog, derivative or structural mimetic.

32. The method of claim 29 wherein the agent is a nucleic acid molecule.

33. The method of claim 32 wherein the nucleic acid molecule is an anti-sense, sense, DNA-derived RNAi or synthetic RNAi directed to the EphA4 receptor mRNA transcript.

34. The method of claim 29 wherein the agent is an antibody or a derivative, recombinant, chimeric or deimmunized form thereof.

35. A method of preventing or reducing the amount of gliosis and/or glial scarring and/or inflammation in the nervous system of a subject said method comprising administering to said subject an effective amount of an antagonist of EphA4-mediated signaling for a time and under conditions sufficient to prevent or decrease gliosis and/or glial scarring and/or inflammation.

36. The method of claim 35 wherein the Eph receptor is the EphA4 receptor.

37. The method of claim 36 wherein the agent is a proteinaceous or non-proteinaceous molecule.

38. The method of claim 35 wherein the agent is a EphA4 antagonist, homolog, analog, derivative or structural mimetic.

39. The method of claim 35 wherein the agent is an ephrin antagonist, homolog, analog, derivative or structural mimetic.

40. The method of claim 35 wherein the agent is a nucleic acid molecule.

41. The method of claim 39 wherein the nucleic acid molecule is an anti-sense, sense, DNA-derived RNAi or synthetic RNAi directed to the EphA4 receptor mRNA transcript.

42. The method of claim 35 wherein the agent is an antibody or a derivative, recombinant, chimeric or deimmunized form thereof.

43. The method of claim 35 wherein the nervous system is the central nervous system (CNS).

44. The method of claim 35 wherein the subject is a human.

45. An isolated agent which is an antagonist of EphA4-mediated signaling for use in reducing gliosis and/or glial scarring and/or inflammation.

46. A pharmaceutical composition comprising the agent of claim 44 and one or more pharmaceutically acceptable carriers and/or diluents and/or excipients.

47. Use of Eph receptor in the manufacture of a medicament for prevention of gliosis and/or glial scarring and/or inflammation.
